(19) United States
(12) Patent Application Publication
(10) Pub. No.: US 2023/0094615 A1
(43) Pub. Date: Mar. 30, 2023

(54) MOBILE DEVICE HAVING MULTIPLE CORRESPONDING SENSORS

(71) Applicant: Huawei Technologies Co., Ltd., Shenzhen (CN)

(72) Inventor: Yurong Xu, Sunnyvale, CA (US)

(73) Assignee: Huawei Technologies Co., Ltd., Shenzhen (CN)

(21) Appl. No.: 18/060,772

(22) Filed: Dec. 1, 2022

Related U.S. Application Data
(63) Continuation of application No. PCT/US2020/070164, filed on Jun. 22, 2020.

(51) Int. Cl. G06F 21/56; G06F 21/55

(52) U.S. Cl. CPC G06F 21/566; G06F 21/552

(57) ABSTRACT

A mobile device such as a smart phone, tablet computer, Internet of things (IoT) device, and/or wearable device includes a sensor module coupled to multiple corresponding sensors. The multiple corresponding sensors include multiple instances of the same type of motion sensor in different physical locations on the mobile device. The sensor module includes a software architecture/algorithm that can configure the multiple corresponding sensors to provide sensor measurements having a higher sampling frequency and/or an improved accuracy. In addition, the sensor module may provide sensor measurements having additional degrees of freedom and/or lower power consumption than can be provided by existing sensor modules. In one example, the multiple corresponding sensors include multiple accelerometers that operate at relatively low power to provide linear acceleration samples, the sensor module processes the multiple linear acceleration samples to generate a measure of angular acceleration used to activate the mobile device.

### PRIORITY CLAIM

This application is a continuation of International Application No. PCT/US2020/070164, filed on Jun. 22, 2020, entitled “MOBILE DEVICE HAVING MULTIPLE CORRESPONDING SENSORS,” the benefit of priority of which is claimed herein, and which application is hereby incorporated herein by reference in its entirety.

### TECHNICAL FIELD

The disclosure generally relates to sensing motion of a smart device and in particular to a sensor system that uses multiple corresponding sensors to provide different types of motion signals.

### BACKGROUND

Mobile computing devices, and especially smart phones, run software programs that sense different types of motion. These devices typically employ a motion sensor such as an inertial measurement unit (IMU) sensor that includes multiple different types of sensors such as one or more accelerometers, one or more gyroscopes, one or more magnetometers as well as other types of sensors such as temperature sensors and pressure sensors. Motion signals sensed by the IMU sensor are provided to different mobile sensing applications running on the mobile device as an operating system (OS) system service. The OS may also use these motion signals to selectively activate and deactivate hardware and/or software elements of the mobile device. The motion signals may also be accessed directly by applications running on the mobile devices using an application program interface (API) such as a sensor hub API. Example mobile applications perform operations such as location based services (LBS), activity recognition, and gesture recognition. Example OS services include IMU-power on, IMU triggered screen saving, and IMU-based augmented reality (AR) or location system services. The sensor hub API may access a hardware sensor hub that includes a low-power processor and memory configured to perform low-level computations based on the sensed motion signals, for example, while the main processor of the mobile device is in a sleep mode. Examples of such computations include step detection, step counting, fall detection, and detection of a device activation gesture. In response to a request from an application, the sensor hub may be configured to accumulate multiple measurements while the main processor is in sleep mode and provide the accumulated measurements to the requesting application when the mobile device awakens from sleep mode.

### BRIEF SUMMARY

The examples below describe apparatus and methods for using a sensor module coupled to multiple corresponding sensors arranged at different locations in the apparatus. The sensor module includes a software architecture/algorithm that can configure the multiple corresponding sensors to provide sensor measurements having a higher sampling frequency and/or an improved accuracy. In addition, the sensor module may provide sensor measurements having additional degrees of freedom and/or lower power consumption than a sensor module using a single sensor.

These examples are encompassed by the features of the independent claims. Further embodiments are apparent from the dependent claims, the description and the figures.

According to a first aspect of the present disclosure a mobile device includes first and second sensors mounted at respectively different locations. The first and second sensors provide respective first and second samples of a first type of motion. A motion processing module of the mobile device obtains the first and second motion samples from the first and second motion sensors and processes the first and second motion samples to provide third motion samples. The motion processing module provides the third motion samples to the mobile device to cause the mobile device to perform an action in response to the third motion samples.

In a first implementation of the mobile device according to the first aspect, the first and second motion sensors include first and second accelerometers, respectively, that provide respective first and second linear acceleration samples as the first and second motion samples. The first and second locations are respectively different locations related to an axis of the mobile device. The motion processing module is to process the first and second linear acceleration samples to calculate, as the third motion samples, a measure of angular acceleration about a pivot point on the axis. The motion processing module is further configured to provide the third motion samples to the mobile device to activate the mobile device.

In a second implementation of the mobile device according to the first aspect, at least one of the first and second motion sensors further includes a gyroscopic sensor configured to provide angular acceleration samples. The motion processing module is configured, in a first mode, to provide the measure of angular acceleration based on the third motion samples and in a second mode to provide the measure of angular acceleration based on the angular acceleration samples.

In a third implementation of the mobile device according to the first aspect, the motion processing module is configured to power-down the gyroscopic sensor when operating in the first mode.

In a fourth implementation of the mobile device, the mobile device further includes an application program interface (API), configured to run on the motion processing module. The API is responsive to a first request type to provide the first motion samples or the second motion samples and is responsive to a second request type to provide the first motion samples and second motion samples.

In a fifth implementation of the mobile device, the motion processing module is configured to combine the first and second motion samples to provide, as the third motion samples, samples indicating the first type of motion and having a signal-to-noise ratio (SNR) that is greater than an SNR of either the first motion samples or the second motion samples.

In a sixth implementation of the mobile device, the motion processing module further comprises selection circuitry, coupled to the first and second motion sensors to selectively provide the first motion samples or the second motion samples in response to a control signal.

In a seventh implementation of the mobile device, the first and second motion sensors are each configured to provide the each of the first and second motion samples at a first sample rate. The motion processing module is configured to provide the control signal to the selection circuitry to repeatedly select the first and second motion samples from the first and second motion sensors at respectively different instants to provide, as the third motion samples, motion samples indicating the first type of motion and having a sample rate greater than the first sample rate.

According to a second aspect, a method for sensing motion of a mobile device obtains first motion samples indicating a first type of motion from a first motion sensor mounted at a first location on the mobile device and obtains second motion samples indicating the first type of motion from a second motion sensor mounted at a second, different, location on the mobile device. The method processes the first and second motion samples to provide third motion samples and provides the third motion samples to the mobile device to cause the mobile device to perform an action in response to the third motion samples.

In a first implementation of the method according to the second aspect, the first and second motion sensors include first and second accelerometers configured to provide respective first and second linear acceleration samples as the first and second motion samples and the first and second locations are respectively different locations related to an axis of the mobile device. The method according to the second aspect processes the first and second linear acceleration samples to calculate, as the third motion samples, a measure of angular acceleration about a pivot point on the axis. The method provides the third motion samples to the mobile device to activate the mobile device.

In a second implementation of the method according to the second aspect, at least one of the first and second motion sensors further includes a gyroscopic sensor configured to provide angular acceleration samples. The method according to the second aspect operates in two modes. In a first mode, the method provides the measure of angular acceleration based on the third motion samples and, in the second mode, the method provides the measure of angular acceleration based on the angular acceleration samples.

In a third implementation of the method according to the second aspect, the method powers-down the gyroscopic sensor when operating in the first mode.

In a fourth implementation of the method according to the second aspect, the method receives, from an application program interface (API) configured to run on the motion processing module. The API is configured to receive a first request to provide the first motion samples or the second motion samples and a second request to provide the first motion samples and second motion samples.

In a fifth implementation of the method according to the second aspect, the method combines the first and second motion samples to provide, as the third motion samples, samples indicating the first type of motion and having a signal-to-noise ratio (SNR) that is greater than an SNR of either the first motion samples or the second motion samples.

In a sixth implantation of the method according to the second aspect, the method selectively provides the first motion samples or the second motion samples in response to a control signal.

In a seventh implementation of the method according to the second aspect, the first and second motion sensors are configured to provide the respective first and second motion samples. The method repeatedly selects the first and second motion samples from the first and second motion sensors at respectively different instants to provide, as the third motion samples, motion samples indicating the first type of motion and having a sample rate greater than the first sample rate.

According to a third aspect, an apparatus for sensing motion of a mobile device, the apparatus includes means for obtaining first motion samples indicating a first type of motion at a first location on the mobile device and means for obtaining second motion samples indicating the first type of motion at a second, different, location on the mobile device. The apparatus further includes means for processing the first and second motion samples to provide third motion samples and means for providing the third motion samples to the mobile device to cause the mobile device to perform an action in response to the third motion samples.

In a first implementation of the apparatus according to the third aspect, the first and second motion samples include first and second linear acceleration samples and the first and second locations are respectively different locations related to an axis of the mobile device. The apparatus further comprises means for processing the first and second linear acceleration samples to calculate, as the third motion samples, a measure of angular acceleration about a pivot point on the axis and the means for providing the third motion samples to the mobile device provides the third motion samples to activate the mobile device.

In a second implementation of the apparatus according to the third aspect, the means for processing the first and second motion samples includes means for combining the first and second motion samples to provide, as the third motion samples, samples indicating the first type of motion and having a signal-to-noise ratio (SNR) that is greater than an SNR of either the first motion samples or the second motion samples.

In a third implementation of the apparatus according to the third aspect, the first and second motion sensors provide the respective first and second motion samples at a first sample rate and the apparatus further includes, means for repeatedly selecting the first and second motion samples from the first and second motion sensors at respectively different instants to provide, as the third motion samples, motion samples indicating the first type of motion and having a sample rate greater than the first sample rate.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

Aspects of the present disclosure are illustrated by way of example and are not limited by the accompanying FIGs. for which like references indicate elements.

FIG. 1 is a perspective drawing of a smart phone mobile device according to an embodiment.

FIG. 2 is a block diagram of a mobile device according to an embodiment.

FIG. 3 is a block diagram of a motion sensor according to an embodiment.

FIG. 4 is a block diagram of a motion processing module according to an embodiment.

FIG. 5 illustrates an example configuration of two motion sensors according to an embodiment.

FIG. 6 is a flow-chart diagram of a method that identifies a wake-up motion based on signals provided by two accelerometers according to an embodiment.

FIGS. 7A, 7B, 7C, 7D, 7E, and 7G are mobile device circuit-board layout diagrams showing positioning of two or more motion sensors according to embodiments.

FIG. 7F is a perspective view of a stacked motion sensor according to an embodiment.

FIG. 8 is a flow-chart diagram of a method implemented by a motion processing module according to an embodiment.

FIG. 9 is a flow-chart diagram of a method for processing samples from multiple motion sensors according to an embodiment.

FIG. 10 is a block diagram of a computing device according to an embodiment.

### DETAILED DESCRIPTION

The embodiments described below implement a motion sensor for a mobile device that includes multiple corresponding motion sensors. The described embodiments can provide increased functionality and/or provide additional sensing features by combining signals from the multiple corresponding motion sensors. In addition, the embodiments describe a software algorithm that is backward-compatible with existing mobile device sensor modules that use a single motion sensor.

As used herein, the term “corresponding motion sensors” and “corresponding sensors” indicates multiple sensors which provide samples that can be used to measure the same type of motion. The corresponding motion sensors may be homogenous sensors (e.g., all linear accelerators) or they can be different types of sensors that provide samples which can be used to measure the same type of motion. For example, as described below, a magnetometer can be used to measure linear acceleration. Consequently, an accelerometer and a magnetometer may be corresponding motion sensors.

An embodiment includes a mobile device such as, without limitation, a smart phone, tablet computer, Internet of things (IoT) device, and/or wearable device, such as augmented reality (AR) glasses, a smart watch, a fall detector, or a health monitor, having a sensor module that includes or is coupled to multiple corresponding sensors. The multiple corresponding sensors include multiple instances of the same type of motion sensor in different physical locations on the mobile device. The example sensor module structure includes a software architecture/algorithm that can configure the multiple corresponding sensors to provide sensor measurements having a higher sampling frequency and/or an improved accuracy. In addition, the sensor module may provide sensor measurements having additional degrees of freedom and/or lower power consumption than can be provided by existing sensor modules.

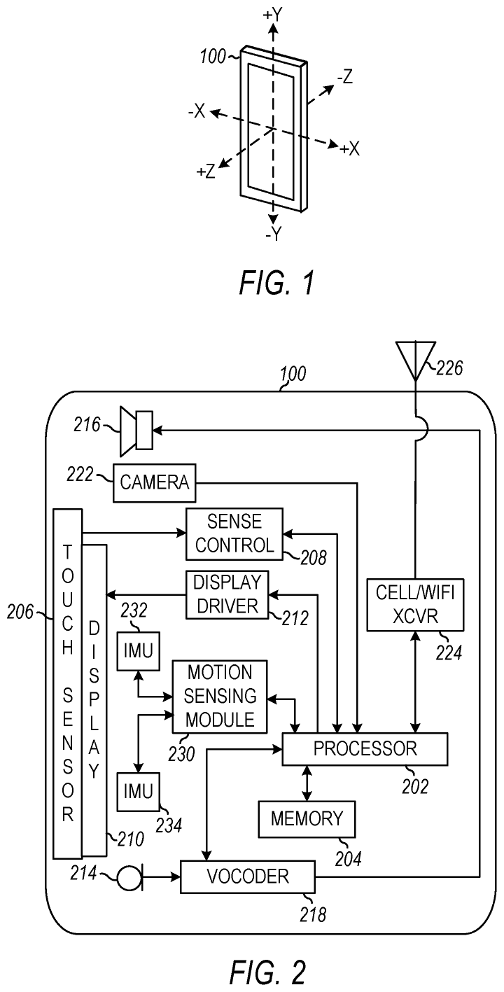

It should be understood that although an illustrative implementation of one or more embodiments is provided below, the disclosed systems, methods, and/or apparatuses described with respect to FIGS. 1-10 may be implemented using any number of techniques, whether currently known or not yet in existence. The disclosure should in no way be limited to the illustrative implementations, drawings, and techniques illustrated below, including the example designs and implementations illustrated and described herein, but may be modified within the scope of the appended claims along with their full scope of equivalents.

In the following description, reference is made to the accompanying drawings that form a part hereof, and in which are shown, by way of illustration, specific embodiments which may be practiced. These embodiments are described in sufficient detail to enable those skilled in the art to practice the inventive subject matter, and it is to be understood that other embodiments may be utilized, and that structural, logical, and electrical changes may be made without departing from the scope of the present disclosure. The following description of embodiments is, therefore, not to be taken in a limiting sense, and the scope of the present disclosure is defined by the appended claims.

The existing sensor hub hardware configuration and API may not be sufficient for future mobile sensing applications. These future mobile sensing applications may use Deep Learning (DL) and Artificial Intelligence (AI) technologies, which may place higher and higher demands on sensors and use more sensory information than can be provided by existing sensing applications. These future applications may perform motion sensing in different ways than are provided by existing sensor hubs. For example, a transportation recognition application may use relatively high frequency sampling of motion signals to identify a specific high-frequency feature of a vehicle. Another application may specify a signal-to-noise ratio (SNR) that is greater than can be provided by an existing sensor hub and IMU.

Existing mobile devices typically use a single sensor solution such as nine-axis IMU sensor. Existing nine-axis IMU sensors, for example the MPU-9250 nine-axis IMU, available from InvenSense® Inc include a set of three accelerometers, a set of three gyroscopic sensors, and set of three magnetometers. Each axis corresponds to a degree of freedom (DOF) of a corresponding sensor. The three sensors in each set are configured to be mutually perpendicular to provide a three DOF acceleration signal, a three-DOF gyroscopic signal, and a three-DOF magnetometer signal. This single IMU architecture, however, may not be able to implement sensing features used in the future applications described above.

Although the embodiments below use a sensor module coupled to multiple motion sensors such as multiple nine-axis IMUs, it is contemplated that the sensor module may be coupled to other sensors, such as one or more temperature sensors and/or pressure sensors, and/or a global navigation satellite system (GNSS) sensor. Furthermore, rather than a nine-axis IMU, the mobile device may use other types of motion sensors, for example one or more single-axis or multi-axis accelerometers or one or more six-axis IMUs, each six-axis IMU including a three-axis accelerometer and a three-axis gyroscope.

FIG. 1 is a perspective drawing of a smart phone mobile device 100 according to an embodiment. FIG. 1 shows an example orientation of X, Y, and Z axes of the smart phone mobile device 100 (referred to hereinafter as mobile device 100). The mobile device 100 is one example mobile device. Other example mobile devices include, without limitation, a tablet computer, a personal digital assistant (PDA), a wearable device, or an IoT device.

FIG. 2 is a block diagram of the mobile device 100 according to an embodiment. The mobile device 100 may function as a digital wireless telephone station. The mobile device 100 includes a display 210 controlled by a display driver 212 coupled to a processor 202. The display 210 serves as an output device for applications running on the processor 202. The mobile device 100 also includes a touch sensor 206 overlaying the display 210 coupled to the processor 202 via a sense control circuit 208. The touch sensor is the input mechanism of the mobile device 100. It is contemplated that other user interface elements may be used such as one or more physical switches, a track ball, or a joystick (none of which are shown in FIG. 2). The mobile device 100 also includes a camera 222 coupled to the processor 202.

The mobile device 100 includes a microphone 214 and speaker 216 which may be used as additional user interface elements for audio input (e.g. audio commands) and output. The microphone 214 and speaker 216 are coupled to a voice encoder/decoder (vocoder) 218 that is coupled to the processor 202 to implement the telephone function in the mobile device 100. For digital wireless communication, the mobile device 100 includes a cellular/Wi-Fi transceiver 224. The transceiver 224 is coupled to the processor 202 and to an antenna 226 to form cellular and/or Wi-Fi connections with base stations, access points, or other devices to, for example, access websites on the Internet.

The processor 202 is also coupled to a memory 204 which may include read-only memory (ROM), flash memory, and/or random access memory (RAM). The memory 204 includes program code for the OS and any applications (Apps) miming on the mobile device 100 as well as data storage for those Apps.

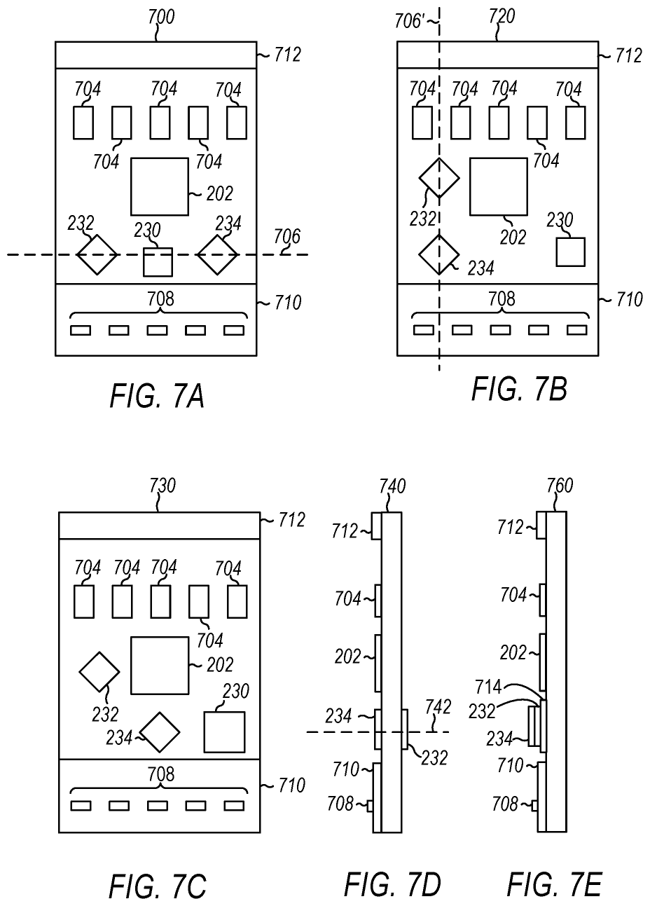

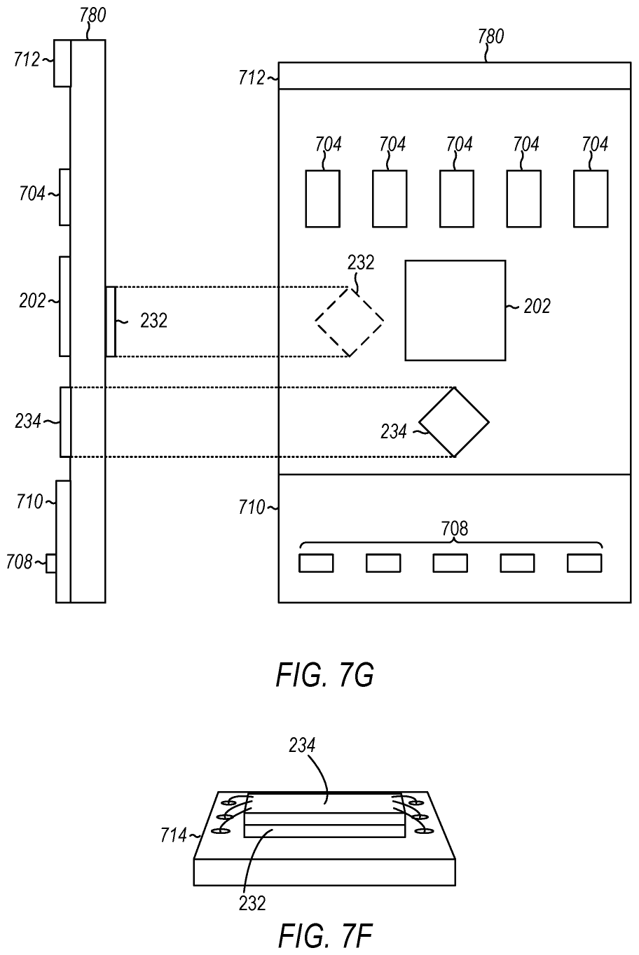

Finally, the mobile device 100 includes a motion processing module 230 that is coupled to multiple IMUs 232 and 234 at different locations in the mobile device 100. While the mobile device 100 includes two motion sensor IMUs 232 and 234, it is contemplated that the mobile device 100 may include more than two motion sensors, as described below with reference to FIGS. 7A-7G. The motion processing module 230 includes processing circuitry that provides functionality comparable to an existing sensor hub. Indeed, as described below, the motion processing module 230 is backward-compatible with existing sensor hubs. The motion processing module 230 may include a microcontroller, coprocessor, or digital signal processor (DSP) that has lower power consumption than the processor 202. Similar to a sensor hub, the motion processing module 230 may be separate from the processor 202 and may include processing circuitry configured to perform low-level computations at low power while the processor 202 and other hardware elements of the mobile device 100 are in a sleep mode. Alternatively, the motion processing module may be implemented by software running on the processor 202 or on an accelerator, sub-processor, or other processing logic (not shown) coupled to the processor 202. The motion processing module 230 is coupled to an interrupt input of the processor 202 to send a wake-up signal to the processor 202 when the motion processing module 230 detects motion of the mobile device 100 corresponding to a predetermined wake-up gesture.

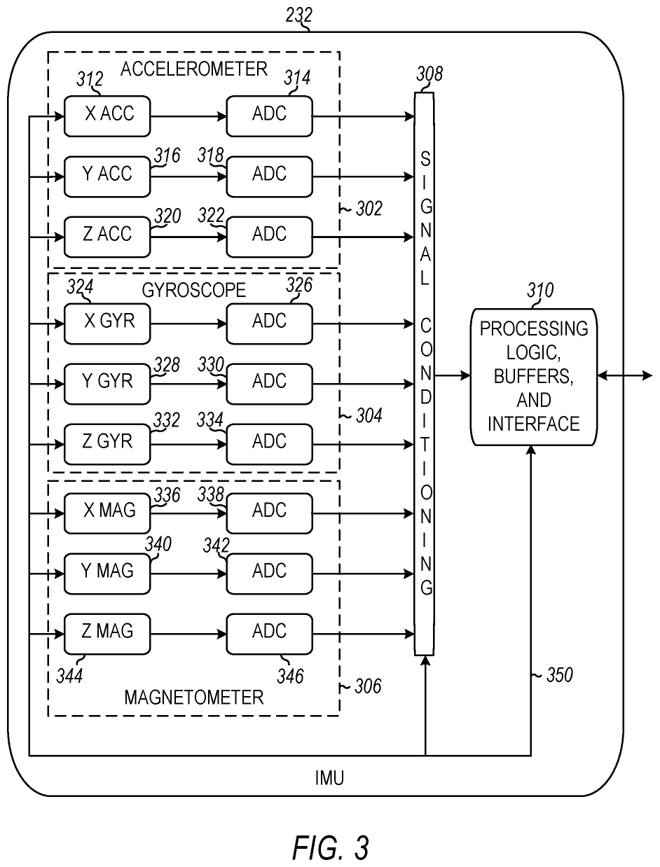

FIG. 3 is a block diagram of an IMU 232 according to an embodiment. The IMU 234 may have the same or a similar configuration. The IMU 232 includes a three-axis accelerometer 302, a three-axis gyroscope 304, and a three-axis magnetometer 306. The three-axis accelerometer 302 includes an X-axis accelerometer 312, a Y-axis accelerometer 316, and a Z-axis accelerometer 320. The three accelerometers 312, 316, and 320 are coupled to respective analog to digital converters (ADCs) 314, 318, and 322. The three-axis gyroscope includes an X-axis gyroscope 324 coupled to an ADC 326, a Y-axis gyroscope 328 coupled to an ADC 330, and a Z-axis gyroscope 332 coupled to an ADC 334. The three-axis magnetometer includes an X-axis magnetometer 336 coupled to an ADC 338, a Y-axis magnetometer 340 coupled to an ADC 342 and a Z-axis magnetometer 344 coupled to an ADC 346. The IMU 232 also includes signal conditioning circuitry 308 and processing logic, buffers, and interface 310. The signal conditioning circuitry 308 filters the output signals provided by the ADCs 314, 318, 322, 326, 330, 334, 338, 342, and 346 to mitigate noise and aliasing distortion. The signal conditioning circuitry 308 also normalizes the output data provided by the ADCs 314, 318, 322, 326, 330, 334, 338, 342, and 346 to compensate for differences in the output ranges of the ADCs 314, 318, 322, 326, 330, 334, 338, 342, and 346. The processing logic, buffers, and interface 310 includes buffer registers for holding results provided by the three-axis accelerometer 302, three-axis gyroscope 304, and/or three-axis magnetometer 306 until the motion processing module 230 requests a sample or sequence of samples. The processing logic, buffers, and interface 310 also includes a bus interface, such as, without limitation, a universal asynchronous receiver-transmitter (UART), an inter-integrated circuit (I²C) interface, and/or a synchronous input-output (SIO) interface. A control bus 350 couples the processing logic, buffers, and interface 310 to each of the accelerometers 312, 316, and 320, each of the gyroscopes 324, 328, and 332, and each of the magnetometers 336, 340, and 344 and to the signal conditioning circuitry 308 to configure the IMU 232 as specified by the motion processing module 230. The bus 350 may be an I²C bus.

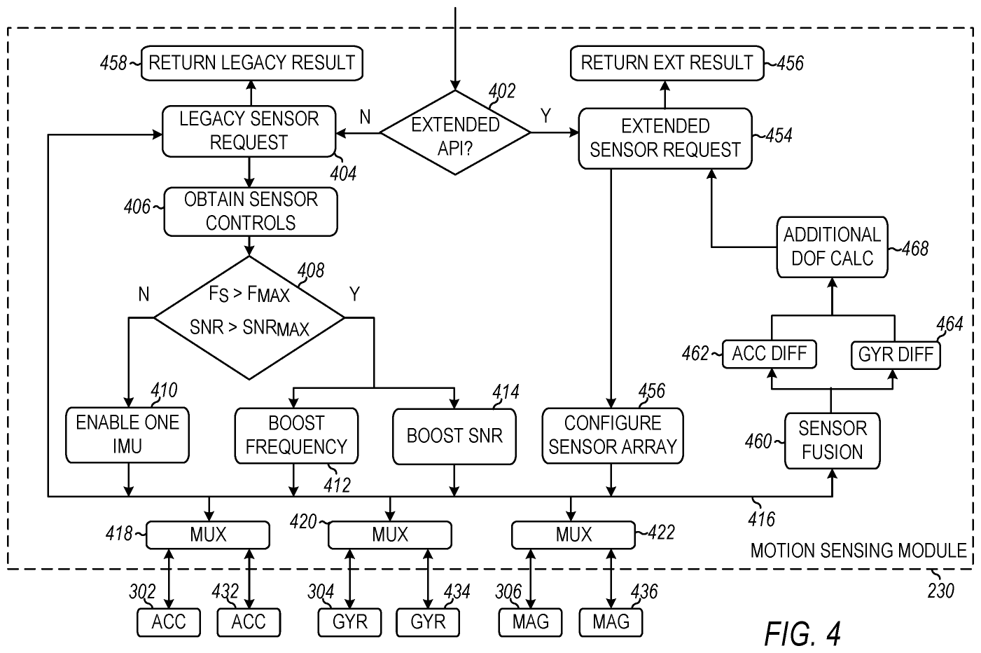

FIG. 4 is a functional block diagram of a motion processing module 230 according to an embodiment. The motion processing module 230 includes additional functionality in addition to the functionality of a sensor hub. Similar to a sensor hub, the motion processing module 230 is implemented using a relatively low-power microcontroller, accelerator, coprocessor, or DSP which is separate from the processor 202 of the mobile device 100. It is contemplated, however, that the functions performed by the motion processing module 230 could be performed by the processor 202.

The motion processing module 230 is coupled, via multiplexers 418, 420, and 422, to two IMUs, a first IMU (e.g., the IMU 232, shown in FIG. 3) and a second IMU (e.g., the IMU 234). The multiplexers 418, 420, and 422 are configured to select samples of different sensed signals from the IMU 232 or the IMU 234 in response to control signals from the motion processing module. The multiplexer 418 is configured to provide control signals to and select acceleration samples from the three-axis accelerometer 302 of the IMU 232 and/or a three-axis accelerometer 432 of the IMU 234. Multiplexer 420 is configured to provide control signals to and select gyroscopic samples from the three-axis gyroscope 304 of the IMU 232 and/or from a three-axis gyroscope 434 of the IMU 234. The multiplexer 422 is configured to provide control signals to and select magnetometer samples from the three-axis magnetometer 306 of the IMU 232 and/or a three-axis magnetometer 436 of the IMU 234. Although FIG. 4 shows the motion processing module 230 being coupled to two nine-axis IMUs, it is contemplated that the motion processing module 230 could be coupled to three or more IMUs and that the IMUs could include fewer sensor components (e.g., a three-axis accelerometer or a six-axis IMU) or more sensor components (e.g., an IMU having a pressure sensor and/or a temperature sensor).

With reference to FIG. 4, the motion processing module 230 is accessed via an API call which requests a sensor measurement at operation 402. The motion processing module 230 returns the result of the sensor measurement via the API at operation 458 or operation 456. As described above, the motion processing module 230 is backward compatible with existing sensor APIs, for example, a legacy sensor hub API. The motion processing module 230, however, provides additional measurements based on the sensed motion. These additional measurements are referred to herein as extended results. At operation 402, the motion processing module 230 receives an API request and determines whether the API request is a legacy sensor request or an extended sensor request. Operation 404 handles legacy sensor requests while operation 454 handles extended sensor requests. From operation 404, the motion processing module 230, at operation 406, obtains the sensor control values from the request. The sensor control values identify a sensor type (e.g., accelerometer, gyroscope, and/or magnetometer) and include parameters for the sensed measurement or measurements. These parameters include, for example, a sampling frequency (F_{S}) and a minimum SNR. Operation 408 determines whether the requested F_{S} is greater than F_{MAX}, the maximum sampling frequency for the requested sensor type, or whether the requested minimum SNR is greater than SNR_{MAX}, the maximum SNR of the requested sensor.

When operation 408 determines that the requested F_{S} is greater than F_{MAX}, it causes operation 412 to boost the frequency of the samples. The boost frequency operation 412 increases the sampling frequency by controlling one of the multiplexers 418, 420, and/or 422 to sample the requested sensor type in different IMUs at different times. For example, when the API request is for an accelerometer measurement, the boost frequency operation 412 controls the multiplexer 418 to alternately select samples from the accelerometers 302 and 432, effectively doubling the sampling frequency relative to sampling a signal accelerometer. Operation 412 also controls the clock signals applied to the accelerometers 302 and 432 so that they are 180 degrees out of phase. When the motion processing module 230 is coupled to more than two IMUs, operation 412 may repeatedly select samples from the multiple IMUs in a round-robin schedule, with appropriately phased clock signals, to achieve a maximum sampling frequency equal to the sampling frequency of a single sensor multiplied by the number of IMUs.

When operation 408 determines that the requested SNR is greater than SNR_{MAX}, operation 414 boosts the SNR of the sampled sensor. Operation 414 concurrently obtains samples from multiple sensors and averages these samples. Averaging pairs of samples increases the SNR of the measurement by, for example, at least 6 dB. Averaging larger numbers of concurrently obtained samples further increases the SNR of the measurement. To increase the SNR of an accelerometer measurement, for example, operation 414 may control the accelerometer 302 and the accelerometer 432 to have the same sampling frequency and sampling phase. Each accelerometer 302 and 432 stores the most recent sample in a register. Operation 414 then controls the multiplexer 418 to successively obtain the most-recently stored samples from the accelerometers 302 and 432 and averages the obtained samples to produce the resulting sample having the increased SNR. In an embodiment in which the motion processing module 230 is coupled to four IMUs, pairs of IMUs may be activated and the operations 412 and 414 may be used together to achieve both a sampling frequency greater than F_{MAX} and an SNR greater than SNR_{MAX}.

When operation 408 determines that the requested F_{S} is not greater than F_{MAX} and that the requested SNR is not greater than SNR_{MAX}, operation 410 enables one IMU, for example, IMU 232, and processes the request in the same way as a legacy sensor hub API. The result of the legacy sensor request is returned via a bus 416 to operation 404 which, in turn, returns the result to the requesting application via operation 458.

When operation 402 determines that the API request is an extended sensor request, the request is handled by operation 454. Operation 454 determines a configuration for the multiple IMUs based on parameters of the request. Operation 456, using bus 416, configures the first IMU 232 and the IMU 234 according to the parameters. Example configurations may select groups of corresponding sensors from the IMUs 232 and 234 for sampling, configure their clock signals and clock signal phases, and configure the multiplexers 418, 420, and/or 422 to provide samples from the selected sensors at time instants determined from the parameters. The parameters of the extended sensor request may combine samples from the groups of corresponding sensors to sense motion that cannot be sensed by one type of sensor alone.

The sensor fusion operation 460 handles the combination of the different groups of sensor samples from different IMUs. One example sensor fusion operation combines data from an accelerometer (e.g., accelerometer 302) with contemporaneous data from a gyroscope (e.g., gyroscope 434) to count steps taken by a user of the mobile device 100 and distinguish the steps from a user riding a bicycle. The gyroscope measures angular acceleration while the accelerometer measures linear acceleration. The angular acceleration of a bicycle rider is similar to the angular acceleration of a walker. The linear acceleration of the bicycle rider is different than the linear acceleration of the walker due to the impact of the walker's feet. The example algorithm uses the accelerometer to detect the impact of the walker's feet while walking and uses the gyroscope to detect angular acceleration. Thus, the fusion of these two types of sensors allows the motion processing module to differentiate between bicycle riding and walking and provide a count of the walker's steps. Another example sensor fusion operation may combine data from an accelerometer with data from a gyroscope to determine whether the sensed motion conforms to the profile for the user falling. In this instance, the gyroscope may detect an angular acceleration conforming to a pivot point at the feet or knees of the user while the accelerometer detects an impact greater than a threshold. This threshold may be set to distinguish between the impact of walking or miming and a larger impact of the user falling.

As described below, some sensor measurements use differences between samples provided by respective corresponding sensors. Operation 462 calculates a difference between the acceleration samples provided by accelerometers 302 and 432 while operation 464 calculates a difference between the gyroscope samples provided by gyroscopes 304 and 434. Although not shown, a similar operation may calculate a difference between the magnetometer samples provided by the magnetometers 306 and 436. Operations 462 and 464 provide these difference samples to an additional DOF calculator 468. As described below, the linear acceleration samples provided by the two accelerometers 302 and 432 may be combined to generate an angular acceleration sample. This is an additional DOF that cannot be calculated from a single linear accelerator. A similar combination of two or more magnetometers may also be used to calculate a measure of angular acceleration. Furthermore, a combination of two or more gyroscopic signals may be used to calculate a centrifugal acceleration or to provide a Coriolis measurement. The results of the extended sensor request are provided to operation 454 to be returned to the requesting application by operation 456.

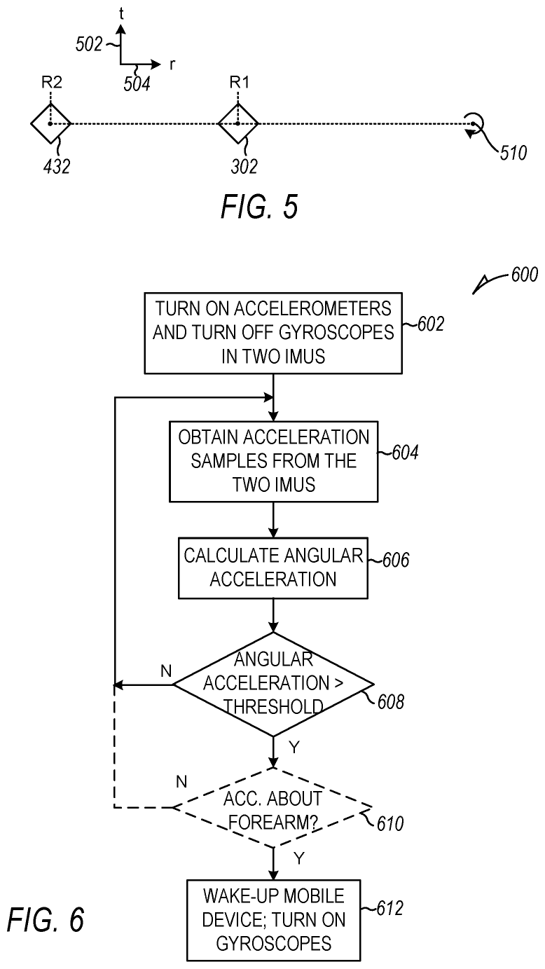

As described above, samples from two corresponding linear accelerometers may be used to calculate a measure of angular acceleration. This example may be used to determine when to awaken the mobile device 100 when it is in a sleep state. Many mobile devices are configured to perform predetermined actions in response to detecting gestures that correspond to the actions. One such gesture is a wake-up gesture in which the user moves the mobile device 100 from a horizontal position to a vertical position to view the display. The mobile device 100 may sense this motion using a sensor hub such that the sensor hub sends a wake-up interrupt to the main processor of the mobile device 100 to cause the mobile device 100 to wake from the sleep state. To sense the wake-up gesture, however, the sensor hub is always powered-on; the sensor hub cannot enter a sleep state. Mobile devices typically use the gyroscope of the IMU to sense angular acceleration. The gyroscope typically uses more operational power than other sensors in the IMU. For example, a gyroscope may use ten times more power than an accelerometer. Consequently, calculating an angular acceleration value using two linear accelerators provides significant power savings for the mobile device 100. FIGS. 5 and 6 describe a method for calculating a measure of angular acceleration based on linear acceleration samples obtained from two spaced accelerometers during a wake-up gesture.

FIG. 5 illustrates an example configuration of the IMUs 232 and 234 according to an embodiment. The IMUs 232 and 234 shown in FIG. 2 arranged at different locations along an r-axis 504 (e.g. corresponding to the Y axis of the mobile device 100 shown in FIG. 1). During the wake-up gesture, the accelerometers 302 and 432 of the IMUs 232 and 234, which are mounted at different locations along the r-axis 504, rotate about a pivot point 510 when the mobile device 100 is rotated from a horizontal position to a vertical position. This results in both of the accelerometers 302 and 432 experiencing acceleration in the direction of the t-axis 502. In this example both of the accelerometers 302 and 432 are mounted on the mobile device 100 and the pivot point is the elbow of the user. Because accelerometer 432 is farther from the pivot point 510 than accelerometer 302, accelerometer 432 experiences greater acceleration during the wake-up gesture.

In the following, α represents the angular acceleration. Two projections of α along the r-axis (e.g., radial components) and the t-axis (e.g., the tangential components) are known from the samples provided by the accelerometers 302 and 432. The value of U can be calculated from these values and from known separation between the two accelerometers 302 and 432. The angular velocity, ω, can be calculated by combining the r-axis acceleration values and the angular acceleration, α, can be calculated by combining the t-axis acceleration values. As shown in FIG. 5, r-axis acceleration experienced by accelerometer 302 is αᵣ₁ and the t-axis acceleration experienced by accelerometer 432 is αᵣ₂. The radial acceleration measured by accelerometer 302 is given by equation (1) while the radial acceleration measured by accelerometer 432 is given by equation (2).

αᵣ₁=ω²R₁ (1)

αᵣ₂=ω²R₂ (2)

The first step in the process is to take the difference between the two measurements, as shown in equation (3).

αᵣ₁−αᵣ₂=ω²(R₁−R₂)=ω²D (3)

where D=R1−R2 is the fixed separation between the accelerometers 302 and 432. From this equation, the magnitude of the angular velocity, ω, can be determined as shown in equation (4).

$$ω = \sqrt{\frac{\vert {α}_{r 2} - {α}_{r 1} \vert}{D}} \qquad ( 4 )$$

Similarly, the tangential accelerations (e.g., t-axis accelerations) measured at accelerometers 302 and 432 are given by equations (5) and (6).

αₜ₁=αR₁ (5)

αₜ₂=αR₂ (6)

The difference between the two measurements is given by equation (7).

αₜ₁−αₜ₂=α(R₂−R₁)=αD (7)

Consequently, the angular acceleration, a is given by equation (8).

$$α = \frac{( {α}_{t 2} - {α}_{t 2} )}{D} \qquad ( 8 )$$

Furthermore, because the two IMUs are mounted at known locations on the mobile device 100, the distance R₁−R₂ is known. The value of Ri can be calculated based on this known distance and the relative values of the tangential accelerations at accelerometers 302 and 432. The value of R₁ may be used to determine whether the angular acceleration is about a pivot point 510 that corresponds to an elbow of the user, or some other motion that indicates that the device should wake-up. A longer value of R₁ may correspond to a pivot point at a shoulder or leg of the user, which might not indicate that the device should wake up. R₁ may also indicate a shorter pivot point than the elbow of the user, a pivot point sensed due to the mobile device 100 vibrating while lying flat on a table, which might also not indicate that the device should wake up. The wake-up gesture is based on movement occurring when the user raises the mobile device to view the screen. In this instance, the elbow pivot point and an angular acceleration greater than a threshold value indicates the wake-up gesture. An R₁ value less than the length of the forearm may be ignored and either of the longer values of R₁ may be used in a health algorithm to count steps taken by the user.

FIG. 6 is a flow-chart diagram of a method 600 that identifies a wake-up motion based on signals provided by the accelerometers 302 and 432 of two IMUs 232 and 234, respectively, according to an embodiment. Operation 602 executes when the mobile device 100 enters a sleep state. This operation powers-on the accelerometers of the 302 and 432 and powers-off the gyroscopes 304 and 434. As described above, the gyroscopes 304 and 434 consume approximately ten times the power of the accelerometers 302 and 432. At operation 604 the method 600 obtains samples from the accelerometers 302 and 432 and at operation 606, the method calculates the angular acceleration from the accelerometer samples as described above with reference to equations (1) to (7). Operation 608 determines whether the measured angular acceleration is greater than an activation threshold. In one embodiment, any angular acceleration greater than the activation threshold activates the mobile device 100 at operation 612. Optionally, as indicated by the dashed-line operation 610, the method 600 may also calculate the value of R₁ and compare the calculated value to the forearm length of the user. Operation 610 may calculate R₁ and compare it to a range of values that capture the variance in forearm length among the general population. Alternatively, R₁ may be compared to a range of forearm lengths determined for a particular user based on data provided by the user, such as the user's height. In this optional implementation, the mobile device 100 is activated when the measured angular acceleration is greater than the threshold and the radius of the acceleration corresponds to the forearm length of the user.

Two spaced magnetometers may be used instead of the two accelerometers to detect the wake-up gesture. In this instance, the two magnetometers would experience different rates of change in magnetic flux as the mobile device is rotated about the pivot point. An analysis similar to that described above may be used to translate the different magnetic flux readings into an angular acceleration measurement.

FIGS. 7A-7G are mobile device circuit-board layout diagrams showing positioning of two or more IMUs according to embodiments. FIGS. 7A-7G describe multiple sensors as being aligned on or parallel to different axes of the mobile device, for example, the X-axis, Y-axis, and Z-axis shown in FIG. 1. FIGS. 7A-7G illustrate different positioning of the multiple sensors to measure multiple different types of motion in multiple DOF. The alignment of the multiple sensors with particular axes of the mobile device is less important than knowledge of the spacing of the multiple sensors and their orientation with respect to the mobile device. Using the known orientation and spacing of the multiple motion sensors, the motion sensing module 230 and/or processor 202, shown in FIG. 2, may be programmed to determine whether the provided motion samples indicate a target motion of the mobile device. As described above, target motions may include, without limitation, a wake-up motion, a walking motion, a cycling motion, and/or a falling motion.

FIGS. 7A-7C and the right-hand portion of FIG. 7G show front-plan views of a circuit board substrate of the mobile device 100 in the same orientation as the mobile device 100 shown in FIG. 1. In FIGS. 7A-7C and the right-hand portion of FIG. 7G, the X-axis is a horizontal line through the center of the substrate along a minor-front axis or a width axis, the Y-axis is a vertical line through the center of the substrate along a major-front axis or a height axis, and the Z-axis is a line through the center of the substrate and coming out of the page along a depth axis. FIGS. 7D, 7E, and the left-hand portion of FIG. 7G show side-plan views of the substrates. In FIGS. 7D, 7E, and the left-hand portion of FIG. 7G, the X-axis is a line through the center of the substrate and coming out of the page, the Y-axis is a vertical line through the center of the substrate, and the Z-axis is a horizontal line through the center of the substrate.

FIG. 7A is a front-plan view of an example layout of a substrate 700 where the IMUs 232 and 234 are arranged along an axis 706 that is parallel to the X-axis of the mobile device 100, as shown in FIG. 1. Although the embodiments shown in FIGS. 7A-7G show the IMUs 232 and 234 mounted on the circuit board substrate, other embodiments may mount one or both of the IMUs 232 and 234 on a housing (not shown) or other component of the mobile device 100. The IMUs 232 and 234 shown in FIG. 7A may be replaced with simple one DOF accelerometers to detect angular acceleration about a pivot point on the X-axis of the mobile device 100 shown in FIG. 1 or an axis parallel to the X-axis shown in FIG. 1. In addition to the IMUs 232 and 234, FIG. 7A the processor 202 and the motion processing module 230 are mounted on the substrate (e.g., circuit board) 700. The substrate 700 includes other integrated circuit devices 704, a set of connector pins 708 on a connector bar 710 used to couple the substrate 700 to the touch sensor 206 and display 210. The substrate 700 also includes a mounting bracket 712 for attaching the substrate 700 to a housing.

FIG. 7B is a front-plan view of a substrate 720 including the elements 202, 230, 704, 708, 710, and 712 described above. Consequently, these elements are not described with reference to FIG. 7B. In addition the substrate 720 includes the two IMUs 232 and 234 arranged along an axis 706′ parallel to the Y-axis of the mobile device 100 as shown in FIG. 1. The accelerometers 302 and 432 of the respective IMUs 232 and 234 may be used, as described above with reference to FIG. 6 to measure angular acceleration of the mobile device 100 around a pivot point on the Y-axis of the mobile device 100.

FIG. 7C is a front-plan view of a layout of a substrate 730 in which the IMUs. 232 and 234 are arranged so that their respective accelerometers 302 and 432 may be used to sense motion along both the X-axis and the Y-axis. The IMUs. 232 and 234 are arranged on an angle to both the X-axis and the Y axis. Accordingly both accelerometers will experience different levels of acceleration when the mobile device 100 is rotated about either or both of the X-axis or Y-axis. Thus, the samples provided by the accelerometers 302 and 432 are related to both the X-axis and the Y-axis. The layout in FIG. 7C includes all of the elements 202, 230, 704, 708, 710, and 712 described above. Thus, these elements are not described with reference to FIG. 7C.

FIG. 7D is a side-plan view of a layout of a substrate 740 in which the two IMUs are arranged to sense different levels of acceleration during rotation of the substrate 740 about a point on an axis 742 parallel to the Z-axis ofthe mobile device 100 as shown in FIG. 1. The side-plan view shows the IMUs 232 and 234 arranged on opposite sides of the substrate 740 along the axis 742. FIG. 7D includes the elements 202, 704, 708, 710, and 712 of the substrate 700, described above with reference to FIG. 7A. The motion processing module 230 is not shown in FIG. 7D for the sake of clarity. In this example, the motion processing module 230 is programmed to calculate angular acceleration based on the known spacing and relative orientation of the two IMUs 232 and 234.

FIGS. 7E and 7F illustrate another Z-axis alignment of the IMUs 232 and 234. FIG. 7E is a side-plan view of a layout of a substrate 760 in which the IMU 234 is mounted on top of the IMU 232 and both of the IMUs 234 and 232 are mounted on a substrate 714 to form a stacked IMU element. The layout shown in FIG. 7E includes the elements 202, 704, 708, 710, and 712 of the substrates described above with reference to FIGS. 7A through 7D. Accordingly, these elements are not described with reference to FIG. 7E. FIG. 7F is a perspective view of the stacked IMU element. In this stacked IMU element, connections to the IMU 232 are made through the substrate 714 while connections to the IMU 234 are made by wire-bonds that couple contacts on the upper surface of the IMU 234 to contact points on the substrate 714 and then, through the substrate 714 to the substrate 760.

FIG. 7G is a combined side-plan view and front-plan view of a layout of a substrate 780 in which two IMUs 232 and 234 are arranged to provide different linear acceleration measurements when the mobile device 100 is rotated about a pivot point on any of the X, Y, or Z axes or any combination thereof. In this configuration, the IMUs 232 and 234 are arranged both on opposite sides of the substrate 780 and on a diagonal to the X and Y axes. Thus, the samples provided by the accelerometers 302 and 432 are related to the X-axis, the Y-axis, and the Z-axis of the substrate 780. The layout shown in FIG. 7G includes the elements 202, 704, 708, 710, and 712 of the substrates described above with reference to FIGS. 7A through 7E. Accordingly, these elements are not described with reference to FIG. 7G. For the sake of clarity, the motion processing module 230 is not shown in FIG. 7G. It is contemplated, however, that it may be placed on either side of the IMU 234 along an axis parallel to the X-axis of the mobile device 100.

Although the example substrate layouts shown in FIGS. 7A through 7E and 7G show two IMUs, 232 and 234, it is contemplated that a mobile device may include three or more IMUs. For example, the layout shown in FIG. 7G could include two additional IMUs (not shown), each mounted opposite one of the IMUs 232 and 234. Alternatively, one or more additional IMUs may be configured on any of the substrates 700, 720, 730, 740, 760, and/or 780 to provide measurements that allow the calculation of additional DOFs and/or provide redundancy in the measurements to reduce Gaussian noise in the samples provided by the .

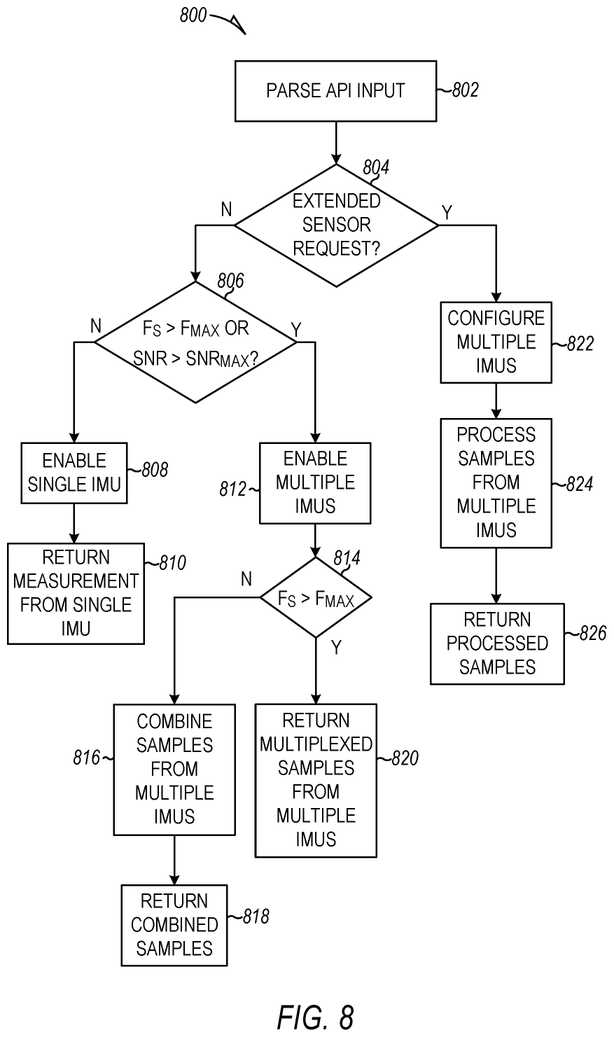

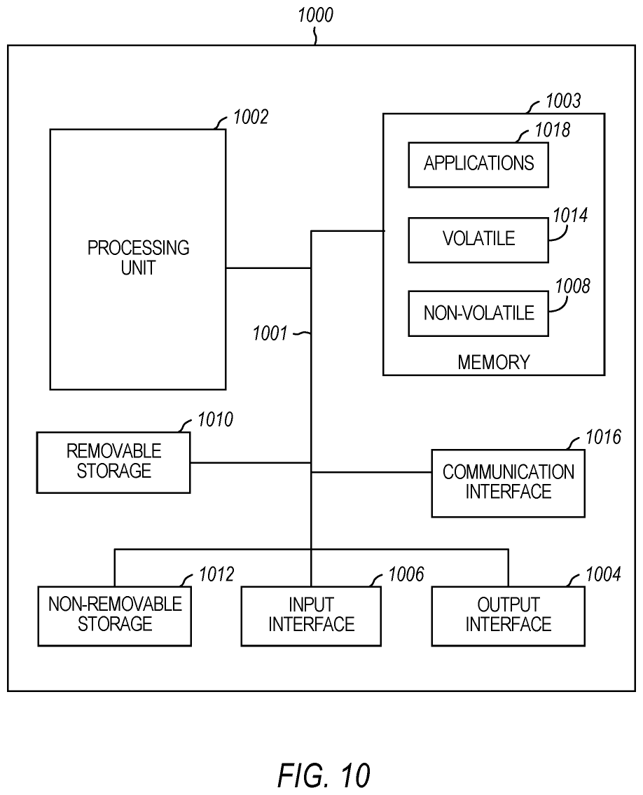

FIG. 8 is a flow-chart diagram showing a method 800 implemented by the motion processing module 230 shown in FIGS. 2 and 4 according to an embodiment. Although the motion processing module 230 in FIG. 4 includes multiple discrete components, it is contemplated that the module may be implemented in software running on a computing device such as the computing device 1000, described below with reference to FIG. 10. As described above, the motion processing module 230 includes the functionality of an existing sensor hub as well as extended functionality. The motion processing module 230 implements an API that is backward compatible with the legacy sensor hub API used by the existing mobile devices. Different mobile devices may have different sensor hub APIs so the materials below describe basic functions performed by the legacy sensor hub API to request and obtain specified sensor measurements from a single IMU.

In FIG. 8, operation 802 receives and parses the input parameters in an API call. Based on the parsed parameters, operation 804 determines whether the API call is a legacy sensor hub request or an extended sensor request. When the call is a legacy sensor hub request, operation 806 determines whether the requested sampling rate, F_{S}, is greater than the maximum sampling rate, F_{MAX} of either of the IMUs 232 or 234. Operation 806 also determines whether the requested SNR is greater than the maximum SNR, SNR_{MAX}, of either of the IMUs 232 or 234. It is noted that although both of the IMUs 232 and 234 include at least one common corresponding sensor (e.g., an accelerometer). The IMUs 232 and 234, however, may not be identical. These devices may not even be IMUs but generic motion sensors. For example one or more of the IMUs 232 or 234 may be a six-axis IMU that does not include a magnetometer or a three-axis accelerometer that does not include a magnetometer or a gyroscope. Accordingly, the IMUs 232 and 234 may have different values for FMAX and SNR_{MAX}. The values of F_{MAX} and SNR_{MAX} used in operation 806 are for the IMU or motion sensor having the larger F_{MAX} and SNR_{MAX}.

When operation 806 determines that the requested F_{S} and SNR are not greater than F_{MAX} and SNR_{MAX}, operation 808 enables one of the IMUs 232 or 234 and obtains the requested measurement or measurements from that IMU. Operation 810 returns the results to the App that initiated the API call.

When operation 806 determines either that the requested F_{S} is greater than F_{MAX} or that the requested SNR is greater than SNR_{MAX}, operation 812 enables multiple IMUs. As described above, it is contemplated that the mobile device may have two or more IMUs. Thus, when the requested F_{S} is greater than F_{MAX} but less than 2*F_{MAX}, operation 812 may enable two IMUs. When the requested F_{S} is greater than N*F_{MAX} but less than (N+1)*F_{MAX}, operation 812 may enable N+1 IMUs. Similarly, when the requested SNR is greater than SNR_{MAX} but less than 2*SNR_{MAX}, operation 812 may enable two IMUs and when the requested SNR is greater than N*SNR_{MAX} but less than (N+1)*SNR_{MAX}, operation 812 may enable N+1 IMUs.

After operation 814 determines that the requested F_{S} is greater than F_{MAX} (or N*F_{MAX}), operation 820 applies appropriately phased clock signals to the enabled IMUs (e.g., IMUs 232 and 234) and controls at least one of the multiplexers (e.g., multiplexers 418, 420, and/or 422) to cycle among the enabled IMUs to provide the requested samples at the requested sampling rate. Operation 820 also returns the samples to the App that initiated the API call.

When operation 814 determines that the requested F_{S} is not greater than F_{MAX} the requested SNR is greater than SNR_{MAX} (or N*SNR_{MAX}). In this instance, operation 816 obtains concurrently sampled measurements from the requested sensor or sensors and averages the concurrently obtained samples. Averaging corresponding samples from two concurrently obtained sample streams improves the SNR of the resulting averaged stream by 6 dB relative to either of the sample streams alone. Averaging samples from more concurrently obtained sample streams provides greater improvement in SNR. After operation 816, operation 818 returns the averaged sample stream to the application that initiated the API call.

When operation 804 determines that the API request is an extended sensor request, operation 822 of the method 800 configures multiple IMUs according to the request. Operation 824 then processes the samples obtained from the multiple IMUs and operation 826 returns the processed samples.

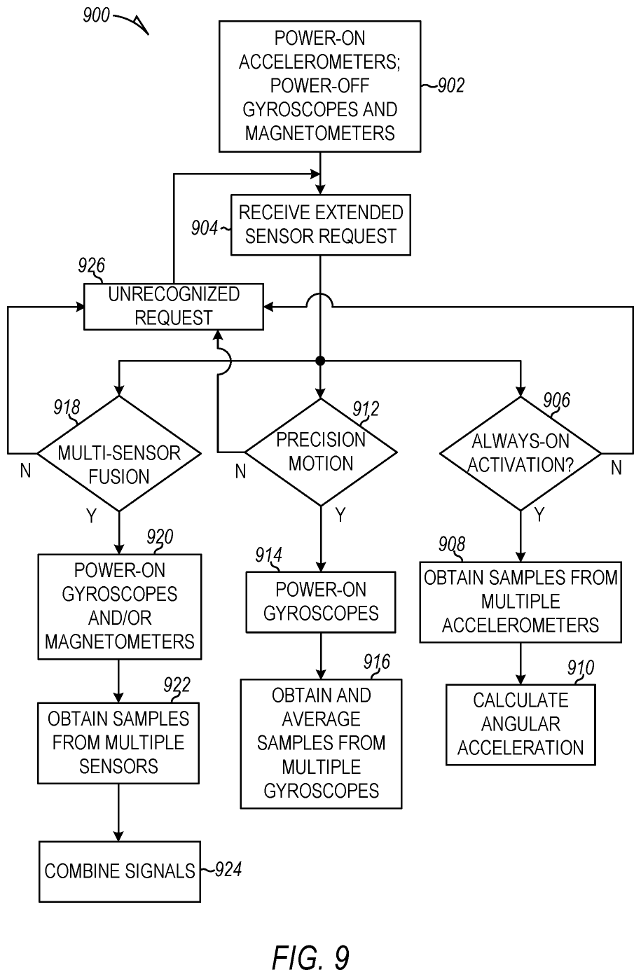

FIG. 9 is a flow-chart diagram of a method 900 for processing samples from multiple IMUs according to an embodiment. The method 900 implements operations 822 and 824 of the method 800. Operation 902 occurs before operation 822. When the mobile device 100 is first powered-up, operation 902 powers-up the accelerometers and powers-down the gyroscopes and magnetometers. Operation 902 initializes the mobile device 100 to operate in always-on mode. Operation 904, corresponding to operation 822, receives the extended sensor request. The extended sensor request is then processed according to one or more of the three paths shown in FIG. 9. These three paths include always-on activation, precision motion, and multi-sensor fusion.

When operation 906 determines that the request is for always-on activation, operation 908 obtains samples from multiple accelerometers and operation 910 processes the samples, as described above with reference to equations (1) to (7) and as shown in FIG. 6, to calculate a measure of angular acceleration based on the accelerometer samples and to determine whether the measure of angular acceleration represents a wake-up gesture.

When operation 912 determines that the request is for high-precision motion samples, operation 914 powers-up the gyroscopes and operation 916 obtains and averages samples from multiple gyroscopes to generate the high-precision motion samples.

When operation 918 determines that the request is to fuse samples from multiple sensors, such as the fusion of gyroscope samples and accelerometer samples to distinguish a walking motion from a cycling motion, operation 920 powers-up the gyroscopes and/or magnetometers of multiple IMUs according to the sensors specified in the API request. Operation 922 obtains the requested samples and operation 924 combines the samples to generate the requested result. After operation 910, 916, or 924, operation 826 of FIG. 8 returns the result to the App that initiated the API call. When the extended sensor request is not recognized by any of operations 906, 912, or 918, operation 926 determines that the request is an unrecognized request and transfers control to operation 904 to wait for the next extended sensor request.

FIG. 10 is a block diagram of a computing device 1000 according to an embodiment. Similar components may be used in the example computing devices described herein. Computing devices similar to computing device 1000 may be used as an alternative to the mobile device 100, shown in FIGS. 1 and 2 and/or may be used to implement the motion processing module 230, shown in FIGS. 2 and 4.

One example computing device 1000 may include a processing unit (e.g., one or more processors and/or CPUs) 1002, memory 1003, removable storage 1010, and non-removable storage 1012 communicatively coupled by a bus 1001. Although the various data storage elements are illustrated as part of the computing device 1000.

Memory 1003 may include volatile memory 1014 and non-volatile memory 1008. Computing device 1000 may include or have access to a computing environment that includes a variety of computer-readable media, such as volatile memory 1014 and non-volatile memory 1008, removable storage 1010 and non-removable storage 1012. Computer storage includes random access memory (RAM), read only memory (ROM), erasable programmable read-only memory (EPROM), electrically erasable programmable read-only memory (EEPROM), flash memory or other memory technologies, compact disc read-only memory (CD-ROM), digital versatile disk (DVD) or other optical disk storage devices, magnetic cassettes, magnetic tape, magnetic disk storage or other magnetic storage devices, or any other medium capable of storing computer-readable instructions. The memory 1003 also includes program instructions for applications 1018 that implement any of the methods and/or algorithms described above.

Computing device 1000 may include or have access to a computing environment that includes input interface 1006, output interface 1004, and communication interface 1016. Output interface 1004 may provide an interface to a display device, such as a touchscreen, that also may serve as an input device. The input interface 1006 may provide an interface to one or more of a touchscreen, touchpad, mouse, keyboard, camera, one or more device-specific buttons, one or more sensors integrated within or coupled via wired or wireless data connections to the server computing device 1000, and/or other input devices. The computing device 1000 may operate in a networked environment using a communication interface 1016. The communication interface may include one or more of an interface to a local area network (LAN), a wide area network (WAN), a cellular network, a wireless LAN (WLAN) network, and/or a Bluetooth® network.

Any one or more of the modules described herein may be implemented using hardware (e.g., a processor of a machine, an application-specific integrated circuit (ASIC), field-programmable gate array (FPGA), or any suitable combination thereof). Moreover, any two or more of these modules may be combined into a single module, and the functions described herein for a single module may be subdivided among multiple modules. Furthermore, according to various embodiments, modules described herein as being implemented within a single machine, database, or device may be distributed across multiple machines, databases, or devices. As described herein, a module can comprise one or both of hardware or software that has been designed to perform a function or functions (e.g., one or more of the functions described herein in connection with providing secure and accountable data access).

Although a few embodiments have been described in detail above, other modifications are possible. For example, the logic flows depicted in the FIGS. 4, 6, 8 and 9 do not require the particular order shown, or sequential order, to achieve desirable results. Other steps may be provided, or steps may be eliminated, from the described flows, and other components may be added to, or removed from, the described systems. Other embodiments may be within the scope of the following claims.

It should be further understood that software including one or more computer-executable instructions that facilitate processing and operations as described above with reference to any one or all of the steps of the disclosure can be installed in and provided with one or more computing devices consistent with the disclosure. Alternatively, the software can be obtained and loaded into one or more computing devices, including obtaining the software through physical medium or distribution system, including, for example, from a server owned by the software creator or from a server not owned but used by the software creator. The software can be stored on a server for distribution over the Internet, for example.

Also, it will be understood by one skilled in the art that this disclosure is not limited in its application to the details of construction and the arrangement of components set forth in the description or illustrated in the drawings. The embodiments herein are capable of other embodiments and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings.

The components of the illustrative devices, systems, and methods employed in accordance with the illustrated embodiments can be implemented, at least in part, in digital electronic circuitry or in computer hardware, firmware, software, or in combinations of them. These components can be implemented, for example, as a computer program product such as a computer program, program code, or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, method, object, or another unit suitable for use in a computing environment. A computer program can be deployed to run on one computer or on multiple computers at one site or distributed across multiple sites and interconnected by a communication network. Method steps associated with the illustrative embodiments can be performed by one or more programmable processors executing a computer program, code, or instructions to perform functions (e.g., by operating on input data and/or generating an output). Method steps can also be performed by, and apparatus for performing the methods can be implemented as, special purpose logic circuitry, for example, as an FPGA (field-programmable gate array) or an ASIC (application-specific integrated circuit), for example.

The various illustrative logical blocks, modules, and circuits described in connection with the embodiments disclosed herein, for example, the motion processing module 230 shown in FIGS. 2 and 4, and the processing logic shown in FIG. 3, may be implemented or performed with one or more general-purpose processors, a digital signal processor (DSP), an ASIC, a FPGA and/or other programmable logic device, discrete gate or transistor logic, discrete hardware components, or any combination thereof designed to perform the functions described herein. A general-purpose processor may be a single core or multi-core microprocessor, but in the alternative, the processor may be any conventional processor, controller, microcontroller, or state machine. A processor may also be implemented as a combination of computing devices, for example, a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random-access memory or both. The elements of a computer include a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, for example, electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof

As used herein, “machine-readable medium” or “computer-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” or “computer-readable medium” should be taken to include a single medium or multiple media (e.g., a centralized or distributed database, or associated caches and servers) able to store processor instructions. A machine-readable medium or computer-readable medium shall also be taken to include any medium (or a combination of multiple media) that is capable of storing instructions for execution by one or more processors, such that the instructions, when executed by one or more processors, cause the one or more processors to perform any one or more of the methodologies described herein. Accordingly, a machine-readable medium or computer-readable medium refers to a single storage apparatus or device, as well as “cloud-based” storage systems or storage networks that include multiple storage apparatus or devices. The term “machine-readable medium” as used herein excludes signals per se.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and could be made without departing from the scope disclosed herein.

Although the present disclosure has been described with reference to specific features and embodiments thereof, it is evident that various modifications and combinations can be made thereto without departing from the scope of the disclosure. For example, other components may be added to, or removed from, the described methods, modules, devices, and/or systems. The specification and drawings are, accordingly, to be regarded simply as an illustration of the disclosure as defined by the appended claims, and are contemplated to cover any and all modifications, variations, combinations or equivalents that fall within the scope of the present disclosure. Other aspects may be within the scope of the following claims.

What is claimed is:

1. A mobile device comprising:
a first motion sensor of a first type coupled to the mobile device at a first location and configured to provide first motion samples indicating a first type of motion;
a second motion sensor of the first type coupled to the mobile device at a second location, different from the first location, and configured to provide second motion samples indicating the first type of motion; and
a motion processing module comprising:
a memory including program instructions; and
one or more processors coupled to the memory, the one or more processors configured to execute the program instructions to perform operations including:
obtaining the first motion samples and the second motion samples from the first motion sensor and the second motion sensor;
processing the first motion samples and the second motion samples to calculate third motion samples; and
providing the third motion samples to an additional processor of the mobile device to cause the mobile device to perform an action in response to the third motion samples.

2. The mobile device of claim 1, wherein:
the first motion sensor includes a first accelerometer configured to provide first linear acceleration samples as the first motion samples;
the second motion sensor includes a second accelerometers configured to provide second linear acceleration samples as the second motion samples;
the operations further comprise processing the first linear acceleration samples and the second linear acceleration samples to calculate, as the third motion samples, a measure of angular acceleration about a pivot point on an axis of the mobile device; and
the action comprises activating the mobile device.

3. The mobile device of claim 2, wherein:
the first motion sensor further comprises a gyroscopic sensor configured to provide angular acceleration samples; and
the motion processing module is configured, in a first mode, to provide the measure of angular acceleration based on the third motion samples and, in a second mode, to provide the measure of angular acceleration based on the angular acceleration samples.

4. The mobile device of claim 3, wherein the motion processing module is configured to power-down the gyroscopic sensor when operating in the first mode.

5. The mobile device of claim 1, further comprising an application program interface (API), configured to run on the motion processing module, the API being responsive to a first request type to provide the first motion samples without providing the second motion samples and being responsive to a second request type to provide the first motion samples and the second motion samples.

6. The mobile device of claim 1, wherein the motion processing module is configured to combine the first motion samples and the second motion samples to provide, as the third motion samples, samples indicating the first type of motion and having a signal-to-noise ratio (SNR) that is greater than an SNR of each of the first motion samples and the second motion samples.

7. The mobile device of claim 1, wherein the motion processing module further comprises selection circuitry, coupled to the first motion sensor and the second motion sensor to selectively provide the first motion samples or the second motion samples in response to a control signal.

8. The mobile device of claim 7, wherein:
the first motion sensor is configured to provide the first motion samples at a first sample rate;
the second motion sensor is configured to provide the second motion samples at a second sample rate; and
the motion processing module is configured to provide the control signal to the selection circuitry to repeatedly select the first motion samples and the second motion samples from the first motion sensor and the second motion sensor at respectively different instants to provide, as the third motion samples, motion samples indicating the first type of motion and having a third sample rate greater than the first sample rate and the second sample rate.

9. A method for sensing motion of a mobile device, the method comprising:
obtaining first motion samples indicating a first type of motion from a first motion sensor mounted at a first location on a mobile device;
obtaining second motion samples indicating the first type of motion from a second motion sensor mounted at a second location on the mobile device, the second location being different from the first location;
processing the first motion samples and the second motion samples to calculate third motion samples; and
providing the third motion samples to a processor of the mobile device to cause the mobile device to perform an action in response to the third motion samples.

10. The method of claim 9, wherein:
the first motion sensor includes a first accelerometer configured to provide first linear acceleration samples as the first motion samples;
the second motion sensor includes a second accelerometer configured to provide second linear acceleration samples as the second motion samples;
the processing of the first motion samples and the second motion samples comprises processing the first linear acceleration samples and the second linear acceleration samples to calculate, as the third motion samples, a measure of angular acceleration about a pivot point on an axis of the mobile device; and
the action comprises activating the mobile device.

11. The method of claim 10, wherein:
the first motion sensor further comprises a gyroscopic sensor configured to provide angular acceleration samples; and
the method further comprises:
providing, in a first mode, the measure of angular acceleration based on the third motion samples; and
providing, in a second mode, the measure of angular acceleration based on the angular acceleration samples.

12. The method of claim 11, further comprising powering-down the gyroscopic sensor when operating in the first mode.

13. The method of claim 9, further comprising:
receiving from an application program interface (API) executing on the mobile device, a first request to provide the first motion samples without the second motion samples; and
a second request to provide the first motion samples and the second motion samples.

14. The method of claim 9, wherein the processing of the first motion samples and the second motion samples comprises combining the first motion samples and the second motion samples to calculate, as the third motion samples, samples indicating the first type of motion and having a signal-to-noise ratio (SNR) that is greater than an SNR of either the first motion samples or the second motion samples.

15. The method of claim 9, further comprising selectively providing the first motion samples or the second motion samples in response to a control signal.

16. The method of claim 15, wherein:
the first motion sensor is configured to provide the first motion samples at a first sample rate;
the second motion sensor is configured to provide the second motion samples at a second sample rate; and
the method further comprises, responsive to the control signal, repeatedly selecting the first motion samples from the first motion sensor and the second motion samples from the second motion sensor at respectively different instants to calculate, as the third motion samples, motion samples indicating the first type of motion and having a third sample rate greater than the first sample rate and the second sample rate.

17. An apparatus for sensing motion of a mobile device, the apparatus comprising:
means for obtaining first motion samples indicating a first type of motion at a first location on a mobile device;
means for obtaining second motion samples indicating the first type of motion at a second location on the mobile device, the second location being different from the first location;
means for processing the first motion samples and the second motion samples to calculate third motion samples; and
means for providing the third motion samples to a processor of the mobile device to cause the mobile device to perform an action in response to the third motion samples.

18. The apparatus of claim 17, wherein:
the first motion samples comprise first linear acceleration samples;
the second motion samples comprise second linear acceleration samples;
the processing of the first motion samples and the second motion samples comprises processing the first linear acceleration samples and the second linear acceleration samples to calculate, as the third motion samples, a measure of angular acceleration about a pivot point on an axis of the mobile device; and
the action comprises activating the mobile device.

19. The apparatus of claim 17, wherein:
the processing of the first motion samples and the second motion samples comprises processing the first motion samples and the second motion samples to calculate, as the third motion samples, samples indicating the first type of motion and having a signal-to-noise ratio (SNR) that is greater than an SNR of either the first motion samples or the second motion samples.

20. The apparatus of claim 17, wherein:
the means for obtaining first motion samples is configured to provide the first motion samples at a first sample rate;
the means for obtaining second motion samples is configured to provide the second motion samples at a second sample rate; and
the apparatus further comprises means for repeatedly selecting the first motion samples and the second motion samples at respectively different instants to calculate, as the third motion samples, motion samples indicating the first type of motion and having a third sample rate greater than the first sample rate and the second sample rate.
